(19) United States
(12) Patent Application Publication
(10) Pub. No.: US 2010/0100026 A1
(43) Pub. Date: Apr. 22, 2010

(54) Wetness sensor utilizing passive resonant circuits

(75) Inventor: Jules Jay Morris, Chestnut Hill, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Fresenius Medical Care Holdings, Inc., Waltham, MA (US)

(21) Appl. No.: 12/288,165

(22) Filed: Oct. 16, 2008

(51) Int. Cl. A61F 13/42; G01M 3/00; A61M 39/02

(52) U.S. Cl. 604/5.04; 73/40; 604/361; 604/4.01

(57) ABSTRACT

An example embodiment of the invention provides a system and method for detecting a liquid leak that includes a sensor with a passive resonant circuit having a resonant frequency, an interrogation unit to transmit a radio frequency (RF) wave corresponding to the resonant circuit's resonant frequency, and a receiver to measure the resonant circuit's response. A determination unit is configured to determine the presence of liquid based on the measured response. In the absence of liquid, the resonant circuit radiates a signal corresponding to its resonant frequency; however, in the presence of liquid, the resonant circuit's electrical characteristics change such that the resonant circuit does not resonate at its resonant frequency. The resonant circuit may include inductive and capacitive elements formed on a substrate using electrically conductive materials. Thus, the system may quickly, safely and accurately detect potentially life-threatening blood leaks that may occur during, for example, hemodialysis.

### BACKGROUND

Devices for detecting the presence of wetness and/or liquid leaks have a number of applications including in mechanical systems having liquid lines or in experimental protocols or devices in laboratories or in use with those undergoing blood treatments. In particular, it is important to detect wetness due to blood leaks or other liquid line leaks during procedures that involve the removal of blood from a person, procedures such as blood donation, blood detoxification, blood filtration/hemofiltration and hemodialysis.

In hemodialysis, for example, blood is removed from a patient through a needle into a blood liquid line circuit that carries the blood to a hemodialysis machine that filters out waste toxins and removes excess water from the blood. Because blood is normally removed from the patient through a blood liquid line circuit at a rapid rate, dislodgement of the needle or a break in the blood liquid line can lead to rapid and potentially fatal blood loss. For this reason, hemodialysis, which generally takes several hours and must be performed several times a week, is typically completed in a medical setting where patients can be supervised. Patients must constantly be monitored visually by medical personnel for blood leaks so that, if needle dislodgement occurs, it can be identified and remedied before detrimental blood loss takes place.

Many devices have been proposed to detect wetness due to liquid leaks, such as blood leaks due to the dislodgement of a needle from a patient or a break/leak in a liquid line; however, these devices have several drawbacks. For example, many known wetness detectors include active electronic circuitry for processing data communications to and from the sensor and may include additional self-test circuitry and/or algorithms necessary to ensure the wetness detector is operating properly. As a result, a local power source, typically a limited life battery, is required to power the detector and its associated circuitry. However, the additional circuitry and related power source increases cost and complexity, and decreases reliability and safety.

In addition, known wetness detectors require excessive amounts of liquid to trigger an alarm or there is too long of a delay between the occurrence and detection of the wetness. In the case of hemodialysis, these deficiencies can be deadly; quick detection and alerting is necessary in order to minimize blood loss due to the dislodgement of a needle or a leak in a liquid line. Further, many proposed or known devices are uncomfortable, unwieldy, unreliable common and relatively expensive.

### SUMMARY

The present invention provides for a liquid leak detection system that can quickly, reliably, and safely detect wetness due to a liquid leak, including blood, and rapidly trigger an alert upon detection of the leak. The liquid leak detection system can be used in a number of situations and is particularly applicable for detecting a blood leak from the dislodgement of a needle in a patient undergoing an extracorporeal blood treatment and/or a liquid leak from a break in a liquid blood line of an extracorporeal blood treatment system, such as a hemodialysis system.

Accordingly, example embodiments of the present invention relate to a liquid leak detection system comprising a sensor including a passive resonant circuit having a resonant frequency where the resonant circuit is configured to sense a presence of liquid. An interrogation unit is configured to transmit a radio frequency (RF) wave or signal corresponding to the resonant frequency of the resonant circuit, and a receiver is configured to measure the resonant circuit's response to the RF wave transmitted by the interrogation unit. A determination unit is configured to determine the presence of liquid based on the measured response.

In one example embodiment, if the resonant circuit senses the absence of liquid, the resonant circuit's response corresponds to its resonant frequency. Conversely, if the resonant circuit senses the presence of liquid, the resonant circuit's response does not correspond to its resonant frequency (e.g., nonexistent or substantially less than).

In other example embodiments, the resonant circuit includes an inductive element electrically connected to a capacitive element where the resonant frequency is a function of the inductive and capacitive values. Electrically conductive materials, such as ink, metal, polymer, silicon, or carbon, may be used to form the inductive and capacitive elements on the surface of a substrate. The inductive and capacitive elements may include an inwardly spiraled conductor connected to an outwardly spiraled conductor that forms a substantially double spiral shaped circuit situated on the substrate. The substrate may comprise a nonconductive material, such as surgical tape, plastic, paper, glass, epoxy resin, or the like. Alternative example embodiments may include multiple sensors associated with multiple patients where each sensor is configured to resonate at different resonant frequencies, thereby allowing the system to identify which sensor is attached to which patient or components (e.g., treatment system, blood lines, etc.) associated with the patient.

In still another example embodiment, the interrogation unit may be configured to transmit the RF wave on a periodic, aperiodic, user initiated, or event driven basis. The sensor may be attached near a blood access site of a patient, such as the patient's skin or an absorbent material surrounding the blood access site.

In response to a determination of the presence of a liquid leak, the determination unit triggers an alert that may include a visual alarm (e.g., flashing light), audible alarm (e.g., siren), physical alert (e.g., vibrating device), warning message displayed on a display, or similar such alert. Alternative example embodiments may include multiple such systems in communication with a central management unit (CMU) where the CMU is configured to monitor the multiple systems, and, in response to a determination of the presence of liquid, issue an alert to identify which sensor, patient, or system component corresponds to the detected liquid.

In yet another example embodiment, the interrogation unit and receiver are integrated with an extracorporeal blood treatment system where the extracorporeal blood treatment includes blood oxygenation, detoxification, transfusion or filtration. For example, the extracorporeal blood treatment system may be a hemodialysis system where the liquid leak detection system directs the hemodialysis system to stop one or more pumps or close one or more blood line valves in response to a determination of a presence of liquid.

Thus, embodiments of the liquid leak detection system of the present invention provide for a technique to easily, rapidly, and accurately detect a liquid leak due to a break in a blood line of a blood treatment system or the disconnect and/or dislodgement of a needle from a needle insertion site. Unlike known wetness sensors, the example wetness sensors disclosed in the present disclosure do not require a local power source (e.g., battery) because the sensor derives all of its operating power from an incoming interrogation pulse. Therefore, in addition to being extremely safe, the example wetness sensors obviate the need for elaborate diagnostics to determine sensor and battery status.

In addition, unlike known sensors that are active in the presence of liquid, example wetness sensors described herein are inactive in the presence of liquid. This provides an additional fail-safe measure in that, if a sensor fails while in operation, the failure (i.e., lack of response) will appear to be a liquid leak and will be quickly identified based on a corresponding alert indicating a leak.

Embodiments of the liquid leak detection system can communicate with multiple wetness sensors and trigger an alarm if a blood leak is detected at any one of the multiple sensors. Moreover, the wetness sensor can be manufactured very inexpensively, effectively allowing the fabrication of disposable sensors, thereby reducing material and maintenance costs as compared with known sensors. Thus, the liquid leak detection system allows treatment systems, in particular, extracorporeal blood treatment systems, to be safer, more reliable, and less costly.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a block diagram illustrating a liquid leak detection system according to an example embodiment of the invention.

FIG. 2A is a schematic diagram illustrating a resonant circuit according to an example embodiment of the invention.

FIGS. 2B and 2C are block diagrams illustrating sensors according to example embodiments of the invention.

FIG. 3 is a block diagram illustrating a liquid leak detection system employing multiple sensors according to an example embodiment of the invention.

FIG. 4 is a block diagram illustrating a liquid leak detection system integrated with a hemodialysis system according to an example embodiment of the invention.

FIG. 5 is a block diagram illustrating a liquid leak detection system employing multiple sensors integrated with multiple hemodialysis systems according to an example embodiment of the invention.

FIG. 6 is a flow diagram of a procedure for detecting a liquid leak performed in accordance with an example embodiment of the invention.

FIG. 7 is a flow diagram of a procedure for detecting a liquid leak performed in accordance with an alternative example embodiment of the invention.

FIG. 8 is a diagram of a computer system that can perform the procedure of FIGS. 6 and 7 in accordance with example embodiments of the invention.

### DETAILED DESCRIPTION

A description of example embodiments of the invention follows.

The present invention relates to a system for detecting the presence of wetness, in particular, wetness due to liquid leakage in a fluid system. Embodiments of the liquid leak detection system comprise a wetness sensor, an interrogation unit, a receiver, and a determination unit. The wetness sensor includes a passive resonant circuit having a resonant frequency where the resonant circuit is configured to sense a presence of a liquid leak. The interrogation unit may transmit a radio frequency (RF) wave or signal corresponding to the resonant frequency of the sensor's resonant circuit. The receiver is configured to measure the resonant circuit's response to the RF wave transmitted by the interrogation unit. The determination unit determines the presence of liquid based on a measured response.

As used herein, the term “liquid leak” refers to any leak, wetness, and/or moisture from the liquid-containing/carrying components of a system (e.g., liquid line, reservoir), generally at a site at which liquid (e.g., blood, water, insulin, antibiotics) is being removed from or returned to and/or infused into a patient, or at a site of interface of those liquid-containing components with other components of the system. Thus, a liquid leak could be a needle insertion site leak and/or a liquid line leak, for example.

To detect liquid leaks, the wetness sensor can be attached at any point to one or more components of a system. For example, the wetness sensor can be in close proximity to a liquid line entry point/needle insertion site or attached to a material surrounding a liquid line entry point/needle insertion site of a liquid line of the system. The liquid line may be a small-sized (e.g., micro-sized) tubing, this type of tubing generally comprised of a polymer (e.g., plastic) having properties (e.g., minimal drag and/or liquid adsorption, non-reactive, non-corrosive, non-degradable) that make it well-suited for carrying the desired liquid.

In a particular embodiment, the system is an extracorporeal blood treatment system in which the wetness sensor may be positioned near a patient's needle insertion site or near various liquid lines of the system to detect leaks in, for example, a hydraulic hose, a line for carrying blood, or, in the case of dialysis, a line for carrying dialysate. The wetness sensor may be situated on a carrier such as adhesive tape, whereby the tape may be securely attached to a desired location. Preferably, the wetness sensor is positioned/placed at connection interfaces of the liquid line, areas of weakness due to their vulnerability to liquid circulation speed, volume, pressure and wear; thus, these connection areas are more prone to leak and, consequently, represent where many leaks occur in practice.

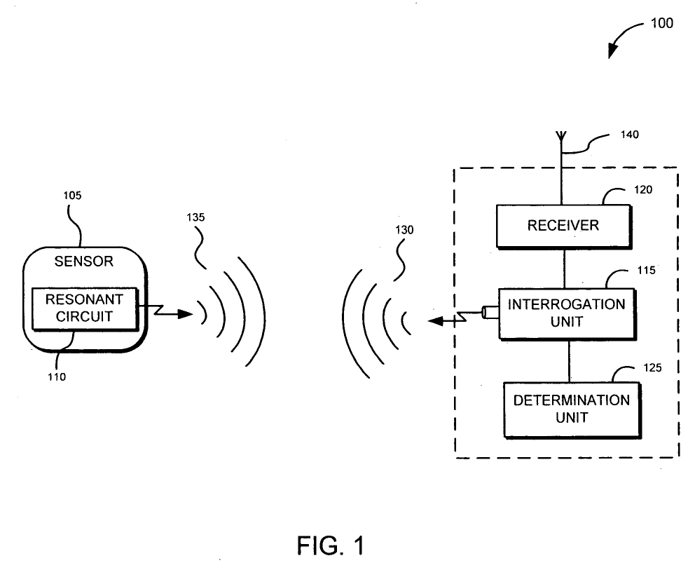

Accordingly, shown in FIG. 1 is a system 100 for detecting a liquid leak in a fluid system that includes a wetness sensor 105, receiver 120, interrogation unit 115, and determination unit 125. The sensor 105 includes a passive resonant circuit 110 that is configured to resonate a signal 135 at a particular resonant frequency. The sensor 105 operates on the principle of resonance where a resonant circuit absorbs and radiates energy (i.e., becomes resonant) when subjected to an appropriate excitation signal.

The resonant frequency is a function of the resonant circuit's 110 electrical characteristics. Altering the resonant circuit's 110 electrical characteristics will change the sensor's 105 resonant frequency. A resonant circuit's 110 electrical characteristics will vary significantly when the resonant circuit 110 is exposed to liquid. Consequently, the resonant circuit 110 will resonate at its resonant frequency when dry and will resonate at a different frequency (if at all) when wet. As a result, the sensor 105 may be employed to detect a liquid leak.

The interrogation unit 115 is configured to transmit an RF signal 130 corresponding to a particular resonant frequency. The interrogation unit 115 may employ an antenna 140 suitable for transmitting RF signals at appropriate frequency and power levels. Multiple different signals may be transmitted by sweeping or stepping through multiple different frequency values. If the transmitted RF signal 130 corresponds to the sensor's 105 resonant frequency, the resonant circuit 110 will absorb and radiated back a signal 135 corresponding to the resonant frequency. Conversely, if the transmitted RF signal 130 does not correspond to the sensor's 105 resonant frequency, the sensor 105 will not respond and will remain dormant.

Thus, when the sensor 105 is in the absence of a liquid leak, the electrical properties of the sensor 105 remained unchanged and the resonant circuit 110 will radiate a signal 135 when excited by an interrogation signal 130 corresponding to its resonant frequency. However, when the sensor 105 is in the presence of a liquid leak, the liquid causes the electrical properties of the sensor's 105 resonant circuit 110 to change such that the resonant circuit's 110 resonant frequency will be substantially different. In this case, when excited by a signal 130 corresponding to the sensor's expected resonant frequency, the sensor 105 will not respond, thereby indicating a liquid leak.

Interrogation unit 115 characteristics, such as transmitted signal frequency and power levels, may be determined based on a number of design objectives including desired operating distance, sensor size, cost, reliability, etc. as well as any regulatory requirements (e.g. FCC), environment requirements (e.g., frequencies approved for hospital or clinical setting) and/or standards body requirements (e.g., UL, CE).

The receiver 120 is configured to measure the resonant circuit's response 135 to RF signals 130 transmitted by the interrogation unit 115. An antenna 140 suitable for receiving RF signals 135 may be in communication with the receiver 120 to improve accuracy and operating distance. The resonant circuit's response 135 may be measured using a number of different techniques known in the art of RF signal communications.

For instance, the receiver 120 may include a frequency selectable power detector tuned to the sensor's 105 resonant frequency to measure the power level of the resonant circuit's response 135. In one example embodiment, the power measurement may be made after energy from the transmitted interrogation signal 130 has been absorbed by the resonant circuit 110. In the absence of a liquid leak, the resonant circuit 110 will radiate energy absorbed from the interrogation signal 130. The resulting power measurement will be equivalent to the power level of the resonant circuit's radiated response 135. However, in the presence of a liquid leak, the resonant circuit's resonant frequency will vary such that the resonant circuit 110 will not absorb energy from the interrogation signal 130. As a result, the resonant circuit 110 will not radiate a signal at a power level corresponding to the expected power level, thereby resulting in a very low measurement value (typically noise). Thus, a relatively low power measurement indicates a liquid leak.

In an alternative embodiment, the power measurement may be made while energy from the interrogation signal 130 is being absorbed by the resonant circuit 110. In this case, in the absence of a liquid leak, the resonant circuit 110 will absorb energy from the interrogation pulse 130 causing the power level measured at the receiver 120 to drop. However, in the presence of a liquid leak, the resonant circuit's 110 resonant frequency will vary such that the sensor 105 will not absorb energy from the interrogation signal 130. In this case, the power level measured at the receiver 120 will be equal to the power level of the interrogation signal 130 as transmitted by the interrogation unit 115. Thus, a relatively high power measurement (i.e., a measured power level that does not drop due to sensor 105 absorption) indicates a liquid leak.

The metrics associated with the measured response are communicated to the determination unit 125 where the determination unit 125 may be configured to determine the presence of liquid based on the metrics provided by the receiver 120. The metrics may be compared to absolute or relative threshold values. For example, the determination unit 125 may compare the determined metrics to metric values expected to be received based on the transmitted interrogation signal 130, and if a metric is above or below a particular threshold value, the determination to 125 may determine the absence or presence of a liquid leak. Upon detection of a liquid leak, the determination unit 125 may issue an alert causing a notification such as an audible or visible warning indicating a leak was detected.

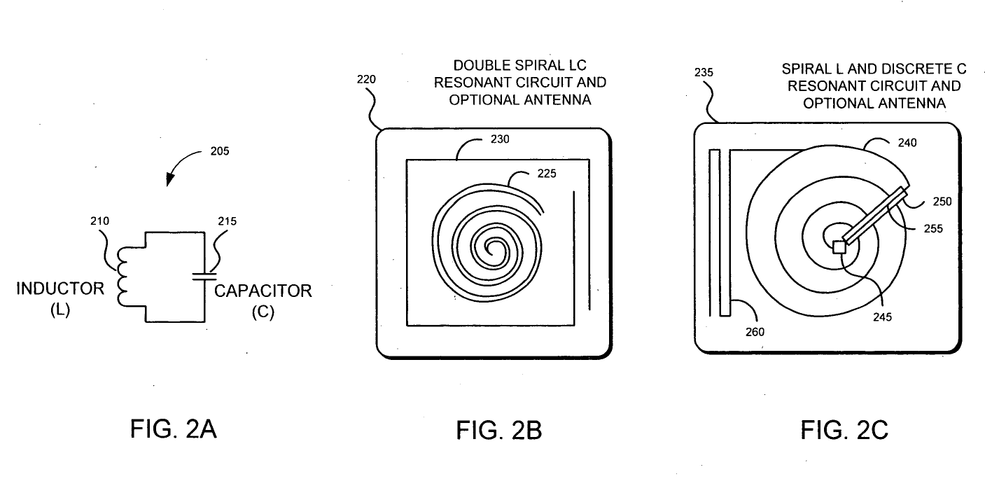

FIG. 2A illustrates a schematic diagram of a passive resonant circuit 205 according to an example embodiment of the present invention. The resonant circuit 205 may include an inductor (L) 210 and a capacitor (C) 215 connected in a parallel configuration where a first side of the inductor 210 is in electrical communication with a first side of the capacitor 215 and a second side of the inductor 210 is in electrical communication with a second side of the capacitor 215 forming a “tuned tank” circuit capable of absorbing energy from an RF interrogation pulse and radiating energy at a resonant frequency corresponding to the circuit's electrical characteristics. It should be noted that alternative parallel/series passive component configurations capable of generating a resonant frequency response when excited by an appropriate interrogation signal may be used as is know in the art of analog circuit design. Inductor 210 and capacitor 215 values may be selected such that the circuit is configured to resonate at a particular resonant frequency where the resonant frequency (fᵣₑₛ) may be calculated using the following formula:

$$fres = \frac{1}{2 π \sqrt{LC}}$$

FIG. 2B is a block diagram of a sensor 220 according to an example embodiment of the present invention. In this embodiment, a resonant circuit is formed using a double spiral LC configuration. A pair of electrical conductors 225 spiral inward toward a center point where they are ultimately connected together. The side-by-side or parallel conductors form a capacitor and the spiral forms an inductor. The sensor may optionally include an antenna 230 that can be, for example, an electrically conductive trace that is formed on the sensor 220. The antenna may allow the sensor to receive and transmit signals over greater distances. This permits additional system flexibility by allowing the sensor 220 and the interrogation unit and receiver to be placed farther apart from each other. Although the antenna 320 is shown as a trace running along the perimeter of the sensor, other geometries such as a zigzag pattern or other configurations know in the art of antenna design may be similarly used.

FIG. 2C is a block diagram of a sensor 235 where a spiral inductor 240 and discrete capacitor 245 are configured to create a resonant circuit according to an alternative example embodiment of the present invention. The inductor 240 is created by forming a single electrical conductor in a spiral configuration. A first side of the inductor 240 (e.g., inner end of the spiral) is connected to a first side of the capacitor 245 and a second side of the inductor 240 (e.g., outer end of the spiral) is connected to a second side of the capacitor 245 using an electrical connector 255 to form a parallel L-C circuit. If the conductor forming the inductor 240 is uninsulated, an insulating layer 250 may be placed between the inductor 240 in the connector 255 to prevent the connector 255 from shorting out the spiral inductor 240.

Alternatively, the second side of the capacitor 245 and the second side of the inductor 240 may be connected to vias (not shown) connecting the capacitor 245 and inductor 240 to the bottom side of the sensor 235. On the bottom side of the sensor 235, the vias associated with the capacitor 245 and inductor 240 may be connected together using a connector similar to connector 255. In this case, the insulating layer 250 may not be necessary. The sensor 235 may also include an optional antenna 260 to similarly extend the signal transmission range allowing the sensor to be placed at a further distance from an interrogation unit and/or receiver. In this case, if the antenna 260 is connected to the inductor 240 as shown in FIG. 2C, the resonant circuit's effective inductance will include any inductance related to the antenna 260. A similar antenna 230 configuration may also be used in conjunction with the resonant circuit shown in FIG. 2B.

The passive wetness sensors depicted in FIG. 2B-C operate on the principle that liquids can be electrically conductive, and, as a result, leaks can be detected using the conductivity of the liquid to which the sensor is exposed. Thus, the sensor can be used to detect any liquid that is electrically conductive, i.e., contains positively and negatively charged ions that enable the liquid to carry electric current. In operation, when a sensor is exposed to an electrically conductive liquid, the liquid will act as a short circuit when in contact with the inductor 225, 240 or capacitor 245. This short circuit will alter the resonant circuit's electrical properties by changing the inductance and/or capacitance values. Because the resonant circuit's resonant frequency is a function of the inductance and capacitance, the liquid will change the resonant circuit's resonant frequency.

The inductive and capacitive elements may be formed using electrically conductive material situated on, for example, the surface of a substrate. The electrically conductive material may include conductive ink, metal, polymer, silicon, carbon, or the like. The substrate may include nonconductive material such as plastic, paper, adhesive tape, or the like. For example, an inductive and capacitive element may be formed using electrically conductive ink printed on the surface of adhesive tape to create an inwardly spiraled conductor connected to an outwardly spiraled conductor effectively producing a double spiral shaped resonant circuit.

It should be noted that the resonant circuits shown in FIGS. 2B and 2C illustrate example embodiments of a sensor employing a resonant circuit. However, the present invention should not be construed as being limited to these specific configurations and other passive resonant circuit configurations known to those skilled in the art may be similarly employed. Furthermore, although the sensors described herein are depicted as including a single resonant circuit, various embodiments may include sensors comprising multiple resonant circuits having the same or different resonant frequencies. For example, a sensor may contain two or more resonant circuits situated thereon to provide an additional measure of redundancy and reliability.

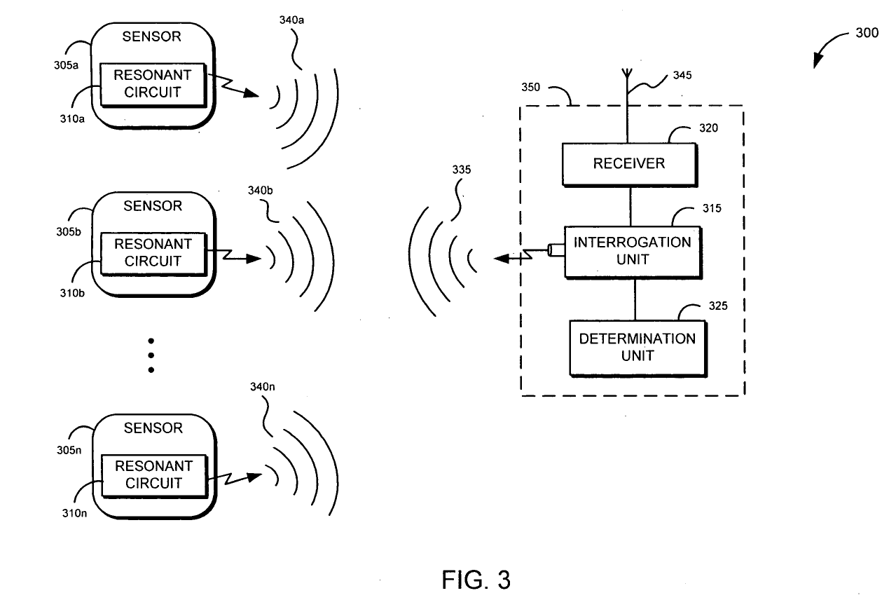

FIG. 3 illustrates an alternative example embodiment of a system 300 configured to simultaneously detect liquid leaks at multiple locations using multiple sensors 305a-n. The system 300 may include a receiver 320 in communication with an antenna 345, interrogation unit 315, determination unit 325, and multiple sensors 305a-n where each sensor 305a-n includes a passive resonant circuit 310a-n. Each sensor 305a-n may be configured to resonate a signal 340a-n at different resonant frequencies with respect to each other, thereby allowing each sensor 305a-n to be individually identified based on its particular resonant frequency response 340a-n.

In this embodiment, the interrogation unit 315 may be configured to sweep or step through multiple frequencies 335 corresponding to each sensor's 305a-n resonant circuit 310a-n to determine whether or not a particular sensor 305a-n senses the presence of a liquid leak. Thus, a single leak detection base unit 350 may be used to simultaneously monitor multiple sensors 305a-n.

In use, the interrogation unit 315 may transmit an RF signal 335 corresponding to the resonant frequency of the first sensor's 305a resonant circuit 310a. In the absence of a liquid leak, the resonant circuit 310a responds by radiating a signal 340a corresponding to its pre-configured resonant frequency. The sensor's response 340a is then received at the leak detection base unit 350 via the antenna 345. The receiver 320 measures and processes the received signal and communicates a metric (e.g., power level) representative of the measured result to the determination unit 325. The determination unit 325 is configured to determine if the metric corresponds with an expected metric associated with the particular resonant circuit 310a being interrogated.

However, if the first resonant circuit 310a is in contact with a liquid, the resonant circuit's 310a electrical characteristics (i.e., inductance and/or capacitance) will change such that the sensor 305a will not radiate a signal corresponding to the expected resonant frequency. The determination unit 325 will determine that the metric representative of the received signal does not match the expected metric(s) associated with the resonant circuit 310a, thereby indicating the presence of a liquid leak.

This technique where 1) the interrogation unit 315 transmits a signal 335 corresponding to the resonant frequency of the sensor under interrogation, 2) the receiver 320 attempts to receive an expected response from a respective resonant circuit 310a-n, and 3) the determination unit 325 determines if the received response 340a-n corresponds with an expected response, may be repeated for each sensor 305a-n in a substantially continuous manner. In this way, each sensor 305a-n may be continuously interrogated so that a liquid leak at any of the sensors may be detected relatively quickly.

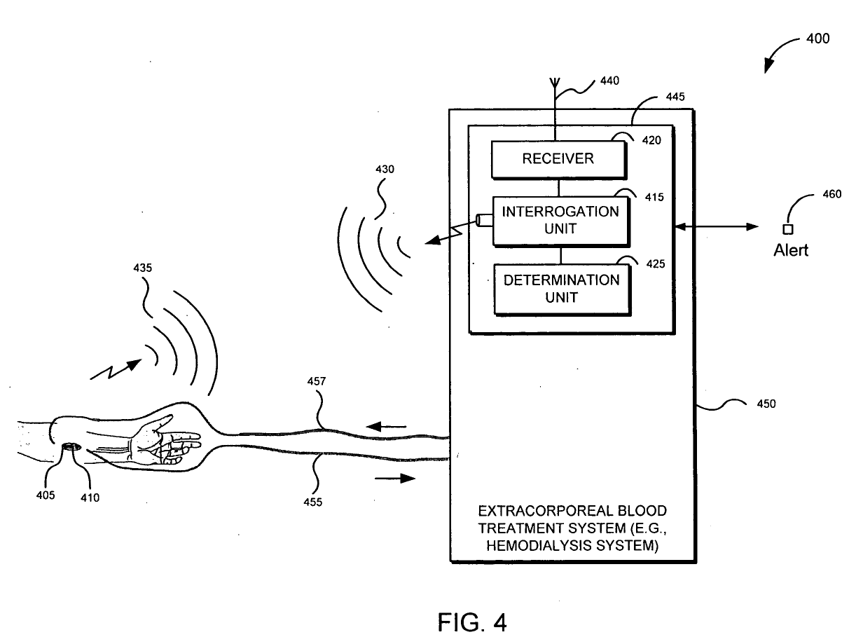

FIG. 4 illustrates a liquid leak detection system 400 used in conjunction with an extracorporeal blood treatment system 450, such as a hemodialysis system, according to an example embodiment of the present invention. The liquid leak detection system 400 includes a leak detection base unit 445 and a sensor 405 comprising a resonant circuit 410. The leak detection base unit 445 may be integrated with the extracorporeal blood treatment system 450 as shown, or alternatively, may be a subsystem in communication with, but separate from the extracorporeal blood treatment system 450, or a combination thereof where one or more of the leak detection base unit's 455 components are integrated with the extracorporeal blood treatment system 450 and one or more of the components are external to the extracorporeal blood treatment system 450.

The leak detection base unit 445 may include an interrogation unit 415, determination unit 425, and receiver 420 in communication with an antenna 440. In a typical extracorporeal blood treatment system 450, blood is removed from a patient using a first needle inserted into the patient's arm or similar location. The blood is transported via a first blood line 455 to the extracorporeal blood treatment system 450 where it is treated. Example treatments include oxygenation, detoxification, transfusion, filtration, or similar such treatments. The treated blood is then transported back to the patient using a second blood line 457 where the blood is returned to the patient's body via a second needle. To reduce the amount of time of the patient must remain attached to the extracorporeal blood treatment system 450, blood is removed at a relatively high rate. However, because of the high rate of blood removal, a life threatening situation can arise should a needle or blood line 455, 457 become dislodged.

In operation, a sensor 405 having a resonant circuit 410 may be placed at, for example, the needle insertion site of a patient about to undergo treatment. The interrogation unit 415 may transmit an interrogation signal 430 corresponding to the resonant circuit's 410 resonant frequency. In the absence of a blood leak, the sensor's 405 resonant circuit 410 radiates a signal 435 corresponding to its resonant frequency. The radiated signal 435 may be detected by the leak detection base unit 445 via the antenna 440 and communicated to the receiver 420. The receiver 420 measures and processes the received signal to determine appropriate metrics, such as the signal's power level at a particular frequency. The metric is communicated to a determination unit 425 to determine if, based on the metric, the received signal was received as expected. If the metrics associated with the received signal 435 corresponds to an expected metric, the determination unit 425 determines that a liquid leak is not present.

If the receiver 420 does not receive a response that corresponds to the expected metric corresponding to the sensor's 405 resonant circuit 410 within an appropriate time period, the determination unit 425 determines that there is a blood leak near the sensor 405 and the leak detection base unit 445 issues an alert signal 460. The determination may be determined by, for example, comparing the metric to a predetermined or programmable threshold. The alert signal 460 may be used to generate an audible or visual alarm, and/or other such indicator(s). Alternatively, or in addition, the alert signal 460 may also be used to cause the extracorporeal blood treatment system 450 to stop one or more blood pumps and/or close one or more valves along the liquid lines to which blood is removed from and/or returned to the patient.

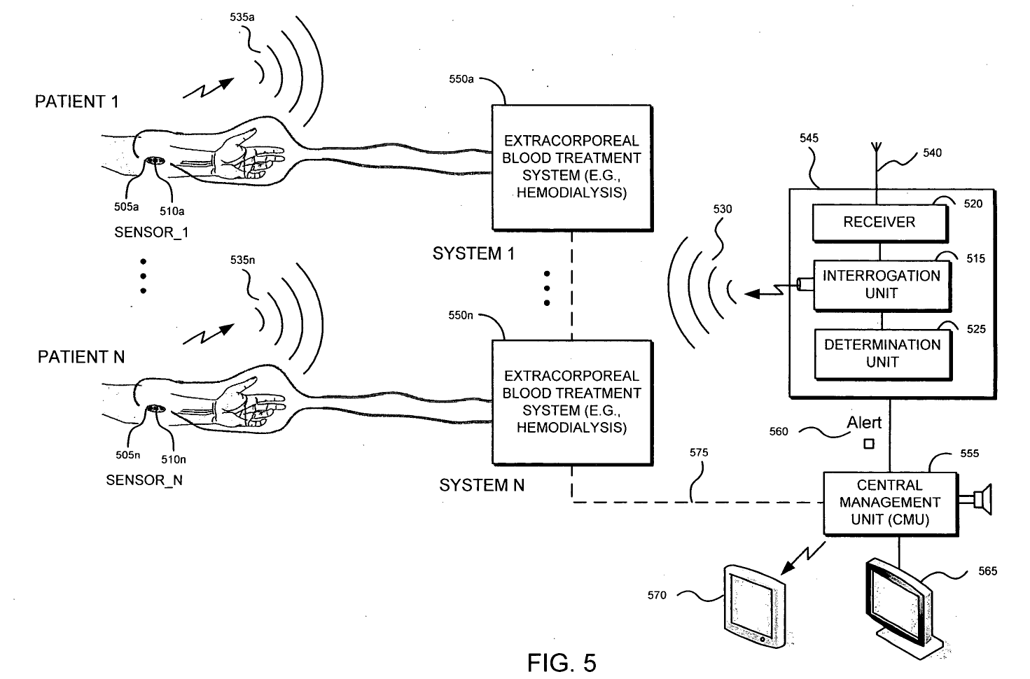

FIG. 5 is a block diagram depicting an example embodiment illustrating a leak detection base unit 545 configured to concurrently monitor multiple sensors 505a-n associated with multiple patients. In a clinical setting, multiple patients may be undergoing blood treatment (e.g., hemodialysis) at the same time. In this embodiment, each sensor 505a-n may be associated with particular patient and the patient's corresponding extracorporeal blood treatment system 550a-n. If a leak is detected in one patient, protective action(s), such as closing pumps or valves on the patient's corresponding extracorporeal blood treatment system 550a-n may be performed without disrupting other patients in which leaks have not been detected.

The leak detection base unit 545 may include a receiver 520, interrogation unit 515, determination unit 525, and antenna 540. The leak detection base unit 545 may be in communication with a central management unit (CMU) 555. The CMU 555 may be in communication with the liquid leak detections base unit 545, multiple extracorporeal blood treatment systems 550a-n, and input/output devices 565, 570 via wired or wireless (or combination thereof) connections 575.

The CMU 555 may be used to coordinate activities between or among the leak detection base unit 545, extracorporeal blood treatment systems 550a-n, sensors 505a-n, and multiple patients. For example, clinical personnel may enter sensor-patient association information via a wired or wireless input device 565, 570. The central management unit 505 may also initiate any appropriate action based on alert messages 560, as well as provide system status information. Note that the CMU 555 may be integrated partially or completely within the leak detection base unit 545 and/or extracorporeal blood treatment systems 550a-n.

Multiple sensors 505a-n may be individually identifiable by configuring each sensor's 505a-n resonant circuit 510a-n to resonate at different unique frequencies. For example, the sensors 505a-n may be configured such that sensor_1 resonates at frequency_1, sensor_2 resonates at frequency_2, sensor_3 resonates at frequency_3, and so on. Each sensor 505a-n may include information representative of its particular resonant frequency printed on the sensor itself, packaging containing the sensor, or similar location.

Before a patient begins treatment, a sensor may be associated with the patient during a patient “registration” procedure. For example, during patient_1's registration, the CMU 555 and/or leak detection base unit 545 may direct an operator to attach a specific sensor (e.g., sensor_1) to the needle insertion site of patient_1. The leak detection unit 545 then associates frequency_1 with the needle insertion site of patient_1. The registration process may also include a leak detection self-test which may be initiated by instructing the operator to simulate a blood leak by, for example, swiping the sensor 505a with their finger or a moistened swab to ensure that the leak detection base unit 545 detects wetness. The sensor 505a is dried and tested again to ensure that it returns to a non-leak detection state by determining that the sensor properly resonates an appropriate signal 535a when interrogated by the interrogation unit 515. Additional sensors may be associated with the patient by repeating this sequence as necessary. After the registration process is complete, the patient's treatment may begin.

The next patient, patient_2, is similarly “registered” whereby the CMU 555 and/or leak detection base unit 545 directs the operator to attach a specific sensor not currently in use (e.g., sensor_2) having a specific resonant frequency (i.e., frequency_2) to the needle insertion site of patient_2. The leak detection base unit 545 associates frequency_2 with the needle insertion site of patient_2. The sensor 505b may be similarly self-tested by performing the leak simulation test described above. Likewise, patient_3 would be similarly “registered” after which the leak detection base unit 545 associates sensor_3's resonant frequency (i.e., frequency_3) with the needle insertion site of patient_3. The association information may be stored in, for example, a lookup table accessible by the leak detection base unit 545 and/or CMU 555. The lookup table may include a list of all sensors available for use with the leak detection system as well as other information including patient, sensor, resonant frequency, treatment system, location, and the like related to sensors currently in use.

Communications between a leak detection base unit 545 and the sensors 505a-n may operate in a manner similar to that described above in conjunction with FIGS. 3 and 4. Thus, the interrogation unit 515 transmits an interrogation signal at frequency_1 and the receiver 520 attempts to receive an expected response from sensor_1. The determination unit 525 determines if the received response 535a corresponds to an expected response. The leak detection base unit 545 will repeat the transmit-receive-determine sequence by continuously sweeping through all the appropriate resonant frequencies for all registered sensors 505a-n currently in use. In an event a leak is detected, the leak detection base unit 545 can identify which particular frequency was not received as expected, and, based on information collected during the registration procedure, the location, patient, and treatment system associated with the leak.

For example, given the preceding example embodiment, should frequency_2 not be received as expected, the leak detection base unit 545 can determine that a leak was detected at the needle insertion site of patient_2. Because a particular frequency is associated with a particular patient and the patient's blood treatment system, safety procedures such as closing valves or pumps on the patient's associated extracorporeal blood treatment system 550a-n may be performed in addition to alerting medical personnel as to which patient the leak was detected.

Furthermore, because the sensors 505a-n do not require a local power source, they may be continuously interrogated without being concerned with draining a battery as is the case with known sensors. The unpowered sensors of the present invention also obviate the need for complicated algorithms to test the battery and related operations, such as placing the sensor in a low power mode to avoid draining the battery.

In an alternative example embodiment, multiple sensors having the same resonant frequency may be registered with a particular patient. For example, identical sensors having the same resonant frequency (e.g. sensor_5/frequency_5) may be placed at patient_1's needle insertion site, extracorporeal blood treatment system 550a, and blood lines. In this case, the expected response would be a “composite” signal representing the sum of responses corresponding to the number of identical sensors currently in use. The expected “composite” signal response may be predetermined, calculated or otherwise derived by the leak detection base unit 545 during the registration procedure. In an event a sensor detects a leak, the received “composite signal” would be different (e.g., decreased power level) such that the determination unit 425 would be able to determine when one or more sensors senses a liquid leak (i.e., does not resonate a signal corresponding to its resonant frequency). Thus, although the location of the particular sensor that detected the leak may not be determined, should any of the sensors detect a leak, the patient's location and associated extracorporeal blood treatment system 550a-n may be identified regardless of which sensor detected the leak.

Although the preceding example embodiments describe attaching a sensor to a patient, sensors may also be attached elsewhere as well. For instance, multiple different sensors may be associated with a patient's respective needle insertion site, extracorporeal blood treatment system, blood lines, and any other location of interest. The patient registration procedure may be performed such that sensor_1/frequency_1 is associated with the needle insertion site of patient_1, sensor_2/frequency_2 is associated with the extracorporeal blood treatment system 550a connected to patient_1, sensor_3/frequency_3 is associated with a blood line(s) used to withdraw blood from patient_1, and sensor_4/frequency_4 is associated with the blood line used to return blood to patient_1. Alternatively, or in addition, multiple sensors may be placed in relatively close proximity (e.g., needle insertion site) to provide redundant sensors at a particular location(s) similar to that described above.

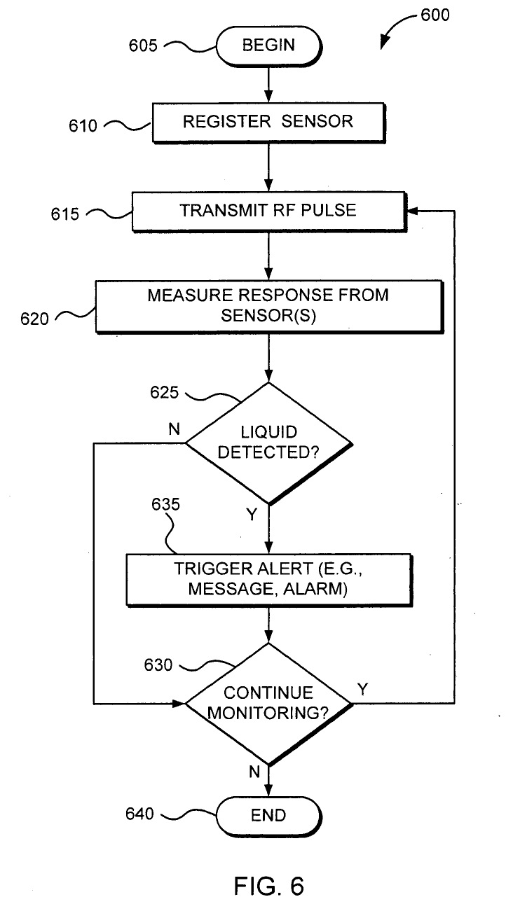

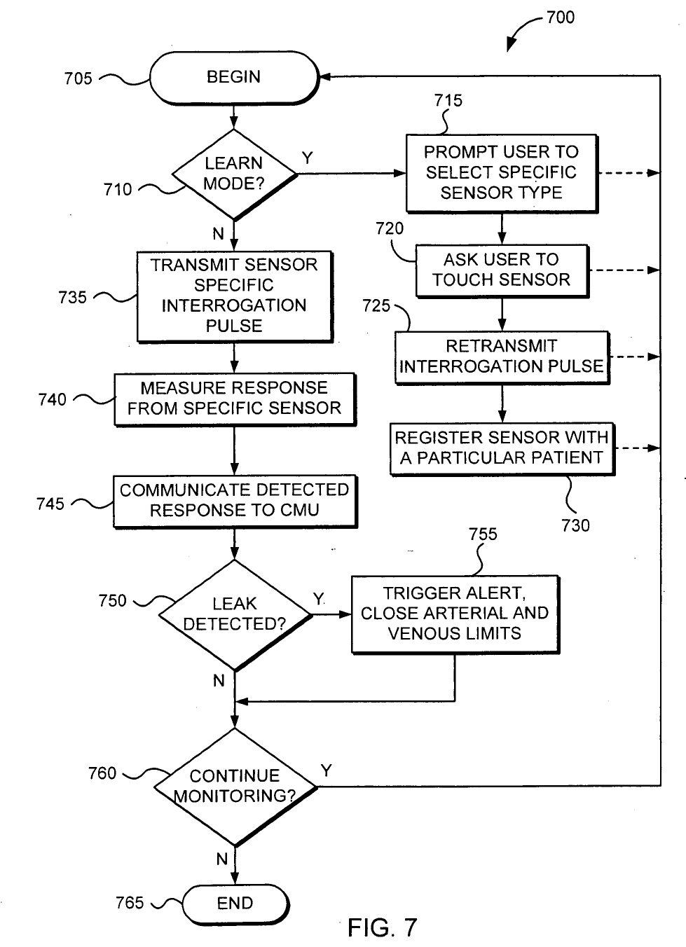

FIGS. 6 and 7 illustrate, in the form of a flow diagrams, example embodiments of the present invention. It should, however, be evident that various modifications and changes may be made thereto without departing from the broader scope of the present invention. For example, some of the illustrated flow diagrams may be performed in an order other than that which is described. It should be appreciated that not all of the blocks illustrated in the flow diagrams are required to be performed, that additional flow blocks may be added, and that some may be substituted with other flow blocks.

FIG. 6 is a flow diagram illustrating a procedure 600 for detecting a liquid leak in a fluid system. Beginning at block 605 the procedure 600 proceeds to block 610 to register a passive resonant circuit having a resonant frequency to sense the presence of a liquid leak. The procedure 600 registers a sensor by, for example, providing and associating a particular sensor with a particular patient, blood treatment system, or blood lines associated therewith. The registration procedure is described below in further detail in conjunction with blocks 715-730 with reference to FIG. 7.

Continuing to refer to FIG. 6, at block 615, the procedure 600 interrogates the sensor by transmitting an RF interrogation pulse that corresponds to the resonant circuit's resonant frequency. The procedure 600 proceeds to block 620, where the sensor's response to the transmitted RF interrogation pulse is measured to determine if the response was received as expected. If at block 625 a leak is detected, the procedure proceeds to block 635 and triggers an alert, such as an alarm, message and/or action and then proceeds to block 630. If a liquid leak was not detected at block 625, the procedure 600 proceeds to block 630. At block 630, the procedure 600 determines if the detection procedure should continue, and if so, the procedure 600 proceeds to block 615 to transmit another RF interrogation pulse. If not, the procedure 600 proceeds to block 640 and ends.

FIG. 7 is a flow diagram illustrating a procedure 700 employing multiple sensors to detect multiple liquid leaks according to alternative example embodiments of the present invention. Such a procedure 700 may be used, for example, at a hemodialysis clinic to centrally monitor blood leaks among multiple patients attached to multiple extracorporeal blood treatment systems.

After beginning at block 705, the procedure 700 determines if learn mode is active at block 710. As a new patient begins the treatment process, learn mode may be used to associate the patient with one or more specific blood leakage sensor(s) prior to treatment. If learn mode is active, the procedure 700 proceeds to block 715 where a user, such as a dialysis technician, may be prompted to select a particular sensor not currently in use. The procedure may maintain a lookup table storing all potential sensor types (i.e., data associated with resonant frequency) available for use with the system. Once a particular sensor type has been placed into use, such information can be recorded in a lookup table. Thus, the procedure 700 may prompt the user to select a sensor not currently in use to ensure that each sensor is uniquely identifiable.

Alternatively, the procedure may prompt the user to manually enter resonant frequency information via a central management unit, the dialysis unit, central or remote interface, or the like. This information may be printed on the sensor itself, on packaging containing the sensor, or similar location. If a sensor with the same resonant frequency has already been placed into service, the central management unit may prompt the user to select a sensor having a different resonant frequency. Alternatively still, the procedure 700 may automatically determine the resonant frequency by, for example, transmitting a low power signal frequency sweep until the sensor's resonant frequency has been determined. Low power interrogation pulses may be used so as not to inadvertently recognize a sensor having the same resonant frequency that has already been placed into service and associated with a different patient. In either case, once the sensor's resonant frequency has been ascertained, an interrogation pulse may be transmitted to the sensor and the sensor's response may be measured to ensure the sensor is working properly.

At block 720 the user may be instructed to simulate a blood leak by applying moisture to the sensor. For example, a user may be instructed to swipe their finger, or a moistened swab, across the surface of the sensor's resonant circuit. The moisture should be sufficient to alter the resonant circuit's characteristic impedance such that when the interrogation pulse is transmitted, the sensor's response will be altered so as not to correspond to the sensor's expected resonant response. After ensuring the sensor detects moisture, the procedure 700 then proceeds to block 725 where, after the sensor has been dried, an interrogation pulse is retransmitted and the sensor's subsequent response is compared to the expected response to ensure the sensor is operating properly. At block 730, the patient being registered is associated with that particular sensor. The sensor and/or patient may also be associated with a particular extracorporeal blood treatment system by prompting the user to enter the appropriate system information. With the registration process complete, the procedure proceeds back to block 705 to begin the procedure 700 using the next sensor (e.g., the next active sensor already in service).

If at block 710, learn mode is not activated, the procedure 700 proceeds to block 735 and transmits an interrogation pulse corresponding to a specific sensor's resonant frequency. At block 740, the procedure 700 measures the sensor's response responsive to the interrogation pulse. At block 745, the detected response may be communicated to a CMU. If at block 750 a blood leak is detected, the procedure 700 proceeds to block 755. At block 755, the procedure 700 may trigger an alert or alarm and/or initiate other safety procedures such as stopping blood pumps and closing arterial and venous valves in the hemodialysis system. If at block 750, a blood leak is not detected, the procedure 700 proceeds to block 760 to determine if monitoring for the presence of blood leaks is to continue, and if not the procedure 700 ends at block 765. If monitoring is to continue at block 760, the procedure 700 proceeds to block 705 where the procedure is repeated again for the next sensor that has been, or is about to, be placed into service.

The procedure 700 may continuously loop relatively quickly such that, even when monitoring multiple patients, the time period between successive monitoring events for each patient is relatively short. As a result, should a leak occur, the leak can be detected in a matter of seconds or less. However, it should be noted that the sequence associated with blocks 715-730, where a new sensor is registered with a patient, may take a few or several minutes to complete. Because of the potential of extended delays during the registration process (e.g., operator delay or non-response), if the procedure continues to wait during a registration procedure delay, a dangerous situation may result should a leak occur in a patient currently undergoing treatment. As a result, the procedure 700 places patients undergoing treatment in a high priority mode with respect to patients undergoing the registration process.

Thus, a parallel processing scheme may be implemented where sensors already placed into service are continually monitored during each of the blocks associated with the registration process. This scheme is represented by the dotted lines extending from blocks 715-730. For example, block 715 may begin by displaying a message prompting the user to select a particular sensor type. However, rather than halting the procedure until the user enters the appropriate information, the procedure may, in parallel, execute a transmit-measure-communicate-detect cycle (i.e. blocks 735-750) for one or more sensors already placed into service. The same parallel monitoring of multiple sensors may occur during any of the remaining registration blocks 720-730.

It should be readily appreciated by those of ordinary skill in the art that the aforementioned blocks are merely examples and that the present invention is in no way limited to the number of blocks or the ordering of blocks described above.

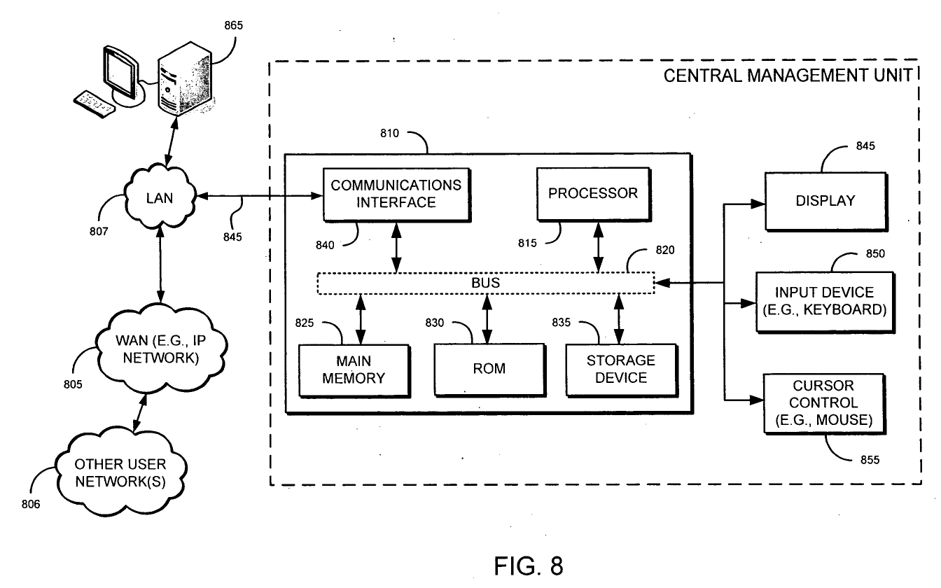

FIG. 8 illustrates a computer system 810 in which example embodiments according to the present invention may be implemented. Components comprising the computer system 810 and associated input/output devices 845, 850, 855 may be integrated within or external to a CMU, within or external to an extracorporeal blood treatment system, or a combination thereof. Information between the computer system 810 and the extracorporeal blood treatment system and/or CMU may be communicated via a direct or networked communications link that may be wired or wireless.

The computer system 810 includes a bus 820 or other communication mechanism for communicating information, and a processor 815 in communication with bus 820 for processing the information. The computer system 810 also includes a main memory 825, such as a random access memory (RAM) or other dynamic storage device, coupled to bus 820 for storing information and instructions to be executed by processor 815. In addition, main memory 825 may be used for storing lookup tables, patient data, a temporary variables, or other intermediate information during execution of instructions to be executed by the processor 815. The computer system 810 may further include a read only memory (ROM) 830 or other static storage device coupled to bus 820 for storing static information and instructions for the processor 815. A storage device 835, such as a magnetic disk or optical disk, is provided and coupled to bus 820 for storing information and instructions.

The computer system 810 may be in communication with a display 845, such as a liquid crystal display (LCD), via bus 820 for displaying information to a user. The display 845 may also provide a touch screen interface such that a user may communicate information to the processor 815 via bus 820 by touching sections of the display 845. An input device 850, including alphanumeric and other keys, may also be in communication with a bus 820 for communicating information and command selections to processor 815. Another type of user input device is cursor control 855, such as a mouse, a stylus, or cursor direction keys for communicating direction information and command selections to processor 815 and for controlling cursor movement on display 845.

Embodiments are related to the use of computer system 810 to control a liquid leakage detection system locally or remotely via the transmission of control messages. According to one example embodiment, control messages related to transmitting interrogation pulses, receiving and measuring corresponding sensor resonant frequency responses, and triggering alerts is provided by computer system 810 in response to processor 815 executing one or more sequences of one or more instructions contained in main memory 825. Such instructions may be read into main memory 825 from another computer-readable medium, such as storage device 835. Execution of the sequences of instructions contained in main memory 825 causes processor 815 to perform the procedures described herein. One or more processors in a multi-processing arrangement may also be employed to execute the sequences of instructions contained in main memory 825. In alternative embodiments, hard-wired circuitry may be used in place of or in combination with software instructions. Thus, embodiments are not limited to any specific combination of hardware circuitry and software.

Further, the hemodialysis or extracorporeal blood treatment system control parameters as well as the instructions to transmit, receive, and analyze resonant frequency responses may reside on a computer-readable medium. The term “computer-readable medium” as used herein refers to any medium that participates in providing instructions to processor 815 for execution. Such a medium may take many forms, including but not limited to, non-volatile media, volatile media, and transmission media. Non-volatile media includes, for example, optical or magnetic disks, such as storage device 835. Volatile media includes dynamic memory, such as main memory 825. Transmission media includes fiber optics, copper wires and coaxial cables, including the wires that comprise bus 820. Transmission media can also take the form of electromagnetic, acoustic, or light waves, such as those generated during RF wave and infrared data communications.

Common forms of computer-readable media include, for example, a floppy disk, a flexible disk, hard disk, magnetic tape, or any other magnetic medium, a CD-ROM, or any other optical medium, RAM, PROM, EPROM, FLASH, or any other memory chip or cartridge, a carrier wave as described hereinafter, or any other medium from which a computer can read.

Various forms of computer readable media may be involved in carrying one or more sequences of one or more instructions to processor 815 for execution. For example, the instructions may initially be carried on a magnetic disk of a remote computer. The remote computer can load the instructions relating to operation of the liquid leakage detection system and/or hemodialysis or extracorporeal blood treatment system remotely into its dynamic memory and send the instructions over a network device such as a router, switch, hub, modem or the like. A network device local to computer system 810 can receive the data on the communications link and use a wireless transmitter to convert the data to a wireless signal. A wireless receiver coupled to bus 820 can receive the data carried in the wireless signal and place the data on bus 820. Bus 820 carries the data to main memory 825, from which processor 815 retrieves and executes the instructions. The instructions received by main memory 825 may optionally be stored on storage device 835 either before or after execution by processor 815.

Computer system 810 also includes a communication interface 840 coupled to bus 820. Communication interface 840 provides a two-way data communication coupling to a network link 845 that is connected to a local network 807. For example, communication interface 840 may be a network interface card to attach to any packet switched local area network (LAN). As another example, communication interface 840 may be an fiber optic line card, asymmetrical digital subscriber line (ADSL) card, an integrated services digital network (ISDN) card or a modem to provide a data communication connection to a corresponding type of communications link or telephone line. Wireless links may also be implemented. In any such implementation, communication interface 840 sends and receives electrical, electromagnetic or optical signals that carry digital data streams representing various types of information.

Network link 845 typically provides data communication through one or more networks to other data devices. For example, network link 845 may provide a connection through local area network 807 to a host computer 865, CMU, or to data equipment operated by a clinic operator, which provides data communication services through the IP network 805 and/or other user network(s) 806. LAN 807 and IP network 805 both use electrical, electromagnetic or optical signals that carry digital data streams. The signals through the various networks and the signals on network link 845 and through communication interface 840, which carry the digital data to and from computer system 810, are exemplary forms of carrier waves transporting the information. Computer system 810 can send control messages and receive data, including program code, through the network(s), network link 845 and communication interface 840.

It should be noted that the computer system 810 may be integrated as part of a hemodialysis or of the extracorporeal blood treatment system, or may be a separate stand-alone unit. In addition, components of the computer system 810 need not be arranged within the same assembly and various components may be connected to various network nodes locally or remotely.

While this invention has been particularly shown and described with references to example embodiments thereof, it will be understood by those skilled in the art that various changes in form and details may be made therein without departing from the scope of the invention encompassed by the appended claims.

What is claimed is:

1. A system for detecting a liquid leak in a fluid system comprising:
a sensor including a passive resonant circuit having a resonant frequency, the passive resonant circuit configured to sense a presence of liquid;
an interrogation unit configured to transmit a radio frequency wave corresponding to the resonant frequency of the resonant circuit;
a receiver configured to measure the resonant circuit response to the radio frequency wave transmitted by the interrogation unit; and
a determination unit configured to determine the presence of liquid based on the measured response.

2. The system according to claim 1 wherein the resonant circuit response corresponds to the resonant frequency if the resonant circuit senses the absence of liquid and corresponds to a frequency substantially less than or greater than the resonant frequency if the resonant circuit senses the presence of liquid.

3. The system according to claim 1 wherein the resonant circuit comprises an inductive element electrically connected to a capacitive element wherein the resonant frequency is a function of the inductive and capacitive values.

4. The system according to claim 3 wherein the inductive and capacitive elements are formed using electrically conductive material.

5. The system according to claim 4 wherein the electrically conductive material is at least one of the following: ink, metal, polymer, silicon or carbon.

6. The system according to claim 4 wherein the inductive and capacitive elements are formed on the surface of a substrate.

7. The system according to claim 6 wherein the inductive and capacitive elements comprise an inwardly spiraled conductor connected to an outwardly spiraled conductor forming a substantially double spiral shape situated on the substrate.

8. The system according to claim 6 wherein the substrate comprises a nonconductive material.

9. The system according to claim 8 wherein the nonconductive material is at least one of the following: surgical tape, plastic, paper, glass or epoxy resin.

10. The system according to claim 1 further including at least two sensors configured to resonate at different resonant frequencies.

11. The system according to claim 10 wherein each sensor is associated with a particular patient.

12. The system according to claim 1 wherein the interrogation unit is configured to transmit the radio frequency wave on a periodic basis.

13. The system according to claim 1 wherein the sensor is attached near a blood access site of a patient.

14. The system according to claim 13 wherein the sensor is attached to an absorbent material surrounding the blood access site.

15. The system according to claim 1 wherein the determination unit triggers an alert in response to a determination of the presence of liquid.

16. The system according to claim 15 wherein the alert is at least one of the following: a display of a warning message, an audible alarm, a visual alarm or a physical alert.

17. The system according to claim 1 wherein at least two such systems are in communication with a central management unit (CMU), the CMU configured to monitor the at least two systems and in response to a determination of the presence of liquid, issue an alert to identify the corresponding sensor.

18. The system according to claim 1 wherein the interrogation unit and the receiver are integrated with an extracorporeal blood treatment system operation unit and the extracorporeal blood treatment is at least one of the following: oxygenation, detoxification, transfusion or filtration.

19. The system according to claim 18 wherein the extracorporeal blood treatment system includes a hemodialysis system.

20. The system according to claim 19 wherein upon a determination of a presence of liquid, the determination unit directs the hemodialysis system to stop one or more pumps or close one or more blood line valves of the hemodialysis system.

21. A method of detecting a leakage in a fluid system, the method comprising:
registering a passive resonant circuit having a resonant frequency to sense a presence of liquid;
transmitting a radio frequency wave corresponding to the resonant frequency of the resonant circuit;
measuring a response generated by the resonant circuit, the response responsive to the transmitted radio frequency wave; and
determining the presence of liquid based on the measured response.

22. The method according to claim 21 wherein the resonant circuit response corresponds to the resonant frequency if the resonant circuit senses the absence of liquid and corresponds to a frequency substantially less than or greater than the resonant frequency if the resonant circuit senses the presence of liquid.

23. The method according to claim 21 wherein the resonant circuit includes an inductive element and a capacitive element and the resonant frequency is a function of the inductive and capacitive values.

24. The method according to claim 21 wherein registering the resonant circuit includes registering at least two resonant circuits to resonate at different resonant frequencies.

25. The method according to claim 24 further including associating each resonant circuit with a particular patient.

26. The method according to claim 25 further including communicating the measured response to a central management unit (CMU), the CMU monitoring the at least two resonant circuits and in response to determining the presence of liquid, issuing an alert identifying the corresponding resonant circuit in which liquid is detected.

27. The method according to claim 21 further including transmitting the radio frequency wave periodically.

28. The method according to claim 21 further including attaching the resonant circuit near a blood access site of a patient.

29. The method according to claim 20 further including attaching the resonant circuit to an absorbent material surrounding the blood access site.

30. The method according to claim 21 further including triggering an alert in response to determining the presence of liquid.

31. The method according to claim 30 wherein triggering the alert includes issuing one or more alarms, the alarms comprising at least one of the following: a displaying a warning message, an audible alarm, a visual alarm or a physical alert.

32. The method according to claim 21 wherein the fluid system is an extracorporeal blood treatment system and wherein the extracorporeal blood treatment is at least one of the following: oxygenation, detoxification, transfusion or filtration.

33. The method according to claim 32 wherein the extracorporeal blood treatment system includes a hemodialysis system.

34. The method according to claim 33 wherein upon determining the presence of liquid, directing the hemodialysis system to stop one or more pumps or close one or more blood line valves of the hemodialysis system.
